(19) United States
(12) Patent Application Publication
(10) Pub. No.: US 2014/0348899 A1
(43) Pub. Date: Nov. 27, 2014

(54) METHOD AND COMPOSITION FOR TREATING CYSTITIS

(71) Applicant: Nanologix Research, LLC, Rancho Mirage, CA (US)

(72) Inventors: Elliot B. Lander, Rancho Mirage, CA (US); Jackie R. See, Fullerton, CA (US)

(73) Assignee: Nanologix Research, LLC, Rancho Mirage, CA (US)

(21) Appl. No.: 13/900,518

(22) Filed: May 22, 2013

(51) Int. Cl. A61K 9/127; A61K 45/06; A61K 38/18; A61K 35/12

(52) U.S. Cl. CPC A61K 9/127; A61K 35/35; A61K 45/06; A61K 38/1808; A61K 38/18
USPC 424/450; 424/537; 514/9.6; 514/7.6

(57) ABSTRACT

A medicament for treating cystitis and a method of treatment are provided. The medicament comprises a stromal vascular fraction provided in a liposomal carrier. Optionally, the medicament also controls one or more additional agents, such as a growth factor, glycosaminoglycan, DMSO, and/or another small molecule used to treat cystitis or having an effect on human stem cells. Cystitis is treated by intravesically administering to a patient a therapeutically effective dose of the medicament. The SVF can be derived from a patient's own adipose cells via liposuction.

### CROSS-REFERENCE TO RELATED APPLICATION

The present application claims priority to and the benefit of U.S. Provisional Application Ser. No. 61/650,393 filed on May 22, 2012, the entire content of which is incorporated herein by reference.

### FIELD OF THE INVENTION

The invention relates to methods of treatment and pharmaceutical compositions for treating various forms of cystitis, in particular the use of liposomal agents as nanocarriers for a stromal vascular fraction for urothelial restorative therapy in cystitis.

### BACKGROUND OF THE INVENTION

Interstitial Cystitis (IC), also known as Interstitial Cystitis/Bladder Pain Syndrome (IC/PBS) is a chronic, severely debilitating, painful condition due to inflammation of the tissues of the bladder wall. The cause is unknown. Symptoms include pelvic pain and pressure, urinary frequency, burning and urgency, and painful intercourse.

IC/BPS is frequently misdiagnosed as a urinary tract infection. Patients often go years without a correct diagnosis. On average, there is about a 4-year delay between the time the first symptoms occur and the diagnosis is made. The condition is usually diagnosed by ruling out other conditions (such as sexually transmitted disease, bladder cancer, and bladder infections). Testing for IC/BPS is not always reliable. The KCl test, also known as the potassium sensitivity test, uses a mild potassium solution to test the integrity of the bladder wall.

The condition generally occurs around age 30 to 50, although it has been reported in younger people. Women are 10 times more likely to have IC/BPS than men. Studies reveal that as many as 3 to 8 million Americans suffer from IC/BPS. The condition is associated with depression, emotional trauma, and other syndromes such as fibromyalgia, endometriosis, and irritable bowel syndrome. Advanced cases may reveal ulcers and erosions in the bladder lining with ultimate scarring and shrinkage of the bladder.

The cause of IC/BPS is unknown. Theories have included neurologic, allergic, autoimmune, toxic exposure, genetic, abnormal mast cells, and psychological. It appears that most patients suffer from a deficiency of the protective glycosaminoglycan (GAG) layer of the inner bladder lining (urothelium). This results in increased permeability of the underlying submucosal tissues with subsequent tissue destruction.

Other forms of cystitis are also known, including hemorrhagic cystitis (including radiation- and chemical-induced cystitis), traumatic cystitis, and chronic cystitis caused by an infectious agent. In each case, the condition is associated with inflammation of the urothelial lining and loss of the glycosaminoglycan (“GAG”) layer to some extent. This causes irritable voiding symptoms including pain, frequency, and urgency. Some of the most common forms of cystitis include:

Radiation Cystitis. This form of cystitis can be disabling and potentially lethal. Radiation-induced degeneration and de-vascularization of the normal urothelium can occur even 10 years after ionizing radiation is delivered to the pelvis for the treatment of malignancy. Radiation cystitis can be hemorhagic. It can be treated with limited effectiveness and is usually incurable.

Chemical Cystitis. This foam of cystitis is often related to the administration of chemotherapy (cytoxan or ifosfamide). These agents can produce acrolein, which has an erosive effect on the urothelium and can cause significant irritative symptoms and even increase the risk for transitional cell carcinoma. Chemical cystitis can be hemorhagic. It can often heal on its own over time.

Chronic Cystitis. Usually bacterial in origin (but can be viral), chronic cystitis is caused by infection. Bacterial infections make the bladder pre-disposed to recurrent infections and severe sensitivity with irritative symptoms. Symptoms can persist for some time even after the active infection is eradicated by appropriate antibiotic therapy. Loss of the GAG layer contributes to reinfection and recurrence of cystitis in an ongoing cycle.

There is no cure for interstitial cystitis, and there are no standard or consistently effective treatments. Treatment is currently based on trial and error and can include opioids, pain inhibitors, antidepressants, vistaril, detrussor relaxants, bladder hydrodistension, bladder instillations (in which a solution is introduced into the bladder via a catheter), biofeedback, dietary modification, and even surgery to enlarge or remove the bladder. Instillations are intravesical treatments typically performed with a number of different combination “cocktails” that may include dimethyl sulfoxide (DMSO), steroids, heparin, chlorpactin, lidocaine, sodium hyaluronate (cystistat), chondroitin (uracyst), and sodium bicarbonate.

Elmiron® (pentosan polysulfate) is the only medication taken by mouth that is specifically approved for treating IC. There have also been reported attempts in the literature at intravesical instillation of Elmiron®. The KCl test has been determined to be helpful in predicting the success of Elmiron®.

Pentosan polysulfate (also known as sodium pentosan polysulfate and pentosan polysulfate sodium) is related to the low molecular weight heparin class of molecules. The official Elmiron® website (www.orthoelmiron.com) states that it is not known exactly how Elmiron® works. Preliminary clinical models suggest that the medicine coats the bladder and the pentosan polysulfate repairs damaged glycosaminoglycan (GAG) layers lining the urothelium. In vitro data suggest that it may provide an anti-inflammatory effect in patients with IC. Pentosan polysulfate shows beneficial effects in a proportion of patients with IC in terms of the improvement of a patient's overall condition and the relief of pain, and it is a generally well tolerated therapy. (Pentosan polysulfate: a review of its use in the relief of bladder pain or discomfort in interstitial cystitis. Anderson V R, Perry C M, Drugs. 2006; 66(6):821-35.) Although most controlled trials suggest a positive effect of oral Elmiron®, some studies have shown little benefit over placebo. It is the only U.S. FDA-approved oral treatment for the relief of bladder pain or discomfort associated with IC. The usual dose is 100 mg taken before or after meals three times per day. A veterinary version of Elmiron® is available under the trademark Cartrophen Vet®.

When administered orally, Elmiron® has pharmacokinetic limitations, as only 6% is absorbed and reaches the circulation, and a mean of 6% of an oral dose is excreted in the urine, mostly as desulfated and depolymerized metabolites. Only a small fraction of the administered dose (mean 0.14%) is recovered as intact drug in urine.

Oral Elmiron® is also associated with several systemic side effects, including hair loss, GI intolerance, headache, rash, sleep disturbance, and vertigo. Rarely, blood thinning can result.

Dr Lowell Parsons, who conducted the original studies on Elmiron®, has also studied intravesical Elmiron® instillation. According to the IC network, an online site that provides information about interstitial cystitis, several preliminary research studies that discussed new instillations were presented at the Bladder Symposium in October 2003, including: #1. Lowell Parsons presented the results for using Elmiron® intravesically. 40 patients were evaluated. 20 received heparin only (40,000 units of heparin) and 20 received Elmiron® (a solution of 100 mg oral Elmiron, 80 mg lidocaine and 3 cc's of sodium bicarbonate). 31 subjects had significant symptom relief. Nine had no change in their symptoms. In response to therapy, there was no significant difference between the two solutions. While heparin and Elmiron® had equal efficacy in the intravesical therapeutic solution, an advantage of pentosan polysulfate over heparin is its substantially lower cost.

There have been several reports in the literature describing the use of liposomes to coat the bladder. See, for example, Tyagi, P., et al., LUTS (2009) 1, S90-S93 (proposing that empty liposomes have a therapeutic effect by forming a coat on the injured urothelium and blocking irritation of submucosal afferent nerves); Lee, W. C., Kaohsiung J Med Sci 2011 Oct. 27 (10): 437-40 (reporting on the safety and dose flexibility clinical evaluation of liposomes in patients with IC, and documenting improvement in symptom scores and side effects); and Tyagi and Chancellor, BJU Int. 2009 December; 104(11):1689-92 (Epub 2009 Jul. 7) (comparing results in rats treated with instillation of liposomes versus intravesical pentosan polysulfate and versus intravesical DMSO. Intravesical liposomes had the most efficacy). Dr. Chancellor and the Lipella Company (www.lipella.com) have described the use of intravesical liposomes to carry the Botulinum toxin into the bladder wall.

Y C Chung et al. in J Urol 2009 October; 182(4): 1393-400 reported on intravesical liposomes versus oral pentosan polysulfate for IC. They found intravesical liposomes achieved efficacy similar to that of oral pentosan polysulfate sodium, and concluded that intravesical liposomes appear to be a promising new treatment for interstitial cystitis/painful bladder syndrome. Some investigators have used empty liposomes to manage IC symptoms and found better results than with instillation of Elmiron® or DMSO. Tyagi P, Hsieh V C, Yoshimura N, Kaufman J, Chancellor M B, “Instillation of liposomes vs dimethyl sulphoxide or pentosan polysulphate for reducing bladder hyperactivity,” British Journal of Urology (BJU Int.) 2009 December; 104(11):1689-92. Epub 2009 Jul. 7. Y C Chung used empty liposomes and proved superior efficacy to oral Elmiron®. Chuang Y C, Lee W C, Lee W C, Chiang P H., J Urol. 2009 Aug. 13. Epub ahead of print. doi:10.1016/j.juro.2009.06.024.

Adipose-derived adult (non-embryonic) mesenchymal stem cells are currently being investigated for use in degenerative conditions that result in damage to various organs and systems. These cells have the ability to seek out areas of injury and regeneration and assist in the repair of nerves, blood vessels, muscle, fat, cartilage, bone, and many other structures. These cells are naturally recruited by cytokines (SDF-1 stromal derived factor one, HGF hepatocyte growth factor, and platelets), to sites of inflammation, ischemia, hypoxia, or injury, and they assist in the healing process either by directly forming needed cells or by secreting chemical messengers that promote healing.

Stem cells are mobilized naturally from bone marrow when the body is healing but they are also found dormant, but available, in human adipose tissue. These stem cells from fat are abundant in levels up to 2500 times greater than those found in bone marrow, and research indicates that the fat-derived stem cells have equivalent regeneration potential to the bone marrow cells (3). Also, stem cell treatment success appears to relate to the number of cells used and this gives adipose cells a significant potential advantage to regenerate human tissues. Mesenchymal stem cells have been used extensively around the world in the successful treatment of orthopedic, cardiac, pulmonary, and neurologic disease in both humans and veterinary models. A recent study in mice with bladder outlet obstruction demonstrated that fluorescent protein-labeled MSC's (mesenchymal stem cells) injected intravenously into test subjects incorporated into bladder muscle resulting in decreased hypoxia, hypertrophy, and fibrosis and increased blood flow. Nine out of ten mice who received MSC's had improved bladder compliance (6).

Intravesical instillation of adipose-derived stem cells into mice effectively shows morphological and phenotypic evidence of smooth muscle incorporation into the bladder wall three months after instillation (8).

The Canadian company, Trillium Therapeutics Inc. (http://www.trilliumtherapeutics.com/therapeutics.html), has investigated the use of recombinant growth factors (HB-EGF) for use in treating IC and other bladder problems.

Despite substantial efforts by the medical community to treat IC and other fauns of cystitis, a truly effective treatment with few side effects has remained elusive.

### SUMMARY OF THE INVENTION

Based on the understanding that adipose-derived stem cells can differentiate into functional smooth muscle cells (4,5), and that patients with IC demonstrate abnormal cell signaling and cytokine release (7), it was determined that stem cell treatment should be helpful for interstitial cystitis patients who exhibit mucosal and smooth muscular damage. A protocol was developed to treat patients with IC of various stages with adipose-derived stem cells. In one embodiment, the protocol uses high doses of stem cells administered intravenously and also intravesically (directly into the bladder lumen). The stem cells can be obtained from the patient using a “mini” liposuction-like procedure performed under local anesthetic. Stem cells are isolated on site from the patient's own fat and then deployed within 90 minutes.

In a first aspect of the invention, a medicament for treating cystitis is provided and comprises a stromal vascular fraction provided in a liposomal carrier. Optionally, one or more additional agents, such as one or more growth factors, variously known as cytokines, chemokines, cellular signaling molecules, leukokines, lymphokines, or interleukins; glycosaminoglycans (GAGs); small molecules like DMSO; and/or or other compounds used in the treatment of IC is also provided in the medicament. In a second aspect of the invention, cystitis is treated by intravesically administering to a patient a therapeutically effective dose of the medicament. Preferably, adult adipose-derived stem cells are also administered intravenously, concurrently with the intravesical administration of the medicament. While not bound by theory, it is believed that the new method of treating cystitis is urothelial restorative therapy, in which the inflammation and degeneration of the bladder urothelial lining is alleviated and the glycosaminoglycan layer of the bladder is restored.

### BRIEF DESCRIPTION OF THE DRAWINGS

FIG. 1 is a print-out from an Invitrogen™ cell counter, presenting a digital image and related data from a patient's SVF containing mesenchymal stem cells, prior to contact with liposomes.

FIG. 2 is a print-out from an Invitrogen™ cell counter, presenting a digital image and related data for the same patient's SVF containing mesenchymal stem cells after being mixed with liposomes, according to the present invention.

### DETAILED DESCRIPTION

In a first aspect of the invention, a medicament for treating cystitis is provided and comprises a stromal vascular fraction (“SVF”) in a liposomal carrier. In one embodiment, the SVF is derived from the patient's adipose cells via liposuction. The stromal vascular fraction (SVF) can be obtained as a product of lipoaspiration (via, e.g., tumescent liposuction), and is rich in mesenchymal (non-embryonic) stem cells (MSCs), preadipocytes, endothelial progenitor cells, T regulatory cells, and anti-inflammatory M2 macrophages.

Optionally, the medicament also contains one or more additional agents, such as one or more growth factors, variously known as cytokines, chemokines, cellular signaling molecules, leukokines, lymphokines, or interleukins; glycosaminoglycans (GAGs); small molecules like DMSO; and/or or other compounds used in the treatment of IC and/or having a physiological effect on stem cells. DMSO has shown some effectiveness in treating IC. Of particular interest is heparin-binding epidermal growth factor (HB-EGF). “TTI-1612” is a recombinant soluble form of HB-EGF in development at Trillium Therapeutics Inc. (Canada). Sigma-Aldrich supplies a number of growth factors and cytokines, and small molecule useful in stem cell biology. A nonlimiting list of growth factors that affect mesenchymal stem cells (MES) is presented in Table 1. A nonlimiting list of small molecules with stem cell effects is presented in Table 2.

TABLE 1
Representative Growth Factors for MES stem cell (Sigma-Aldrich)

Bone morphogenetic protein 2 human
Bone morphogenetic protein 4 human
Bone morphogenetic protein 7 human
Epidermal growth factor human
Fibroblast growth factor-basic human
Fibroblast growth factor-basic from bovine pituitary
Fibroblast growth factor-basic heparain stabilized human
Granulocyte-macrophage colony-stimulating factor mouse
Granulocyte colony-stimulating factor human
Granulocyte-macrophage colony-stimulating factor human
Hepatocyte growth factor human
Insulin-like growth factor-I-human
Insulin-like growth factor-I from mouse
Interferon-γ human
Interferon-γ from mouse
Leukemia inhibitory factor from mouse
Leukemia inhibitory factor human
Platelet-derived growth factor AA human
Platelet-derived growth factor BB human
Transforming growth factor-α human
Transforming growth factor-β1 human
Transforming growth factor-β1 from porcine platelets
Transforming growth factor-β2 from porcine platelets
Transforming growth factor-β2 human
Transforming growth factor-β3 human
Vascular endothelial growth factor human

TABLE 2
Representative small molecules (Sigma-Aldrich)

| Molecule | Stem cell-related activity |
|---|---|
| All-trans retinoic acid | Induces stem cell differentiation |
| Trichostatin A | Histone deacetylase inhibitor |
| BIX 01294 | Histone methyltransferase inhibitor |
| Forskolin | Adenylyl cyclase inhibitor |
| Ascorbic acid | Induces stem cell differentiation |
| Geldanamycin | HSP90 inhibitor |
| 5-Azacytidine | Induces stem cell differentiation |
| Q511 | ARFGAP1 inhibitor; activates |
| | Wnt/β-catenin signaling |
| Cyclopamine | Hedgehog signaling pathway inhibitor |
| 16,16-Dimethyl- | Signaling molecule |
| prostaglandin E2 | |
| Dexamethasone | Induces stem cell differentiation |
| LY-294,002 | Specific cell permeable |
| | phosphatidylinositol 3-kinase inhibitor |
| Myoseverin | Reversible inhibitor of tubulin |
| | polymerization |
| Reversine | Reverses differentiation toward |
| | multipotency in some cell types |
| Y-27632 | ATP-competitive ROCK inhibitor |

GAGs, also known as mucopolysaccharides, are long, unbranched polysaccharides consisting of a repeating disaccharide unit: a hexose or a hexuronic acid linked to a hexosamine. Both sulfated and unsulfated forms are known. Nonlimiting examples include chondroitins (e.g., chondroitin sulfate), dermatans (dermatan sulfate), heparans (heparan sulfate), heparins, hyaluronans (hyaluronic acid, hyaluronates), keratans (keratan sulfate), and pentosans (e.g., pentosan polysulfate). Also included are physiologically acceptable acid, base, ester, and salt forms of such compounds, and mixtures thereof. GAGs such as pentosan polysulfate, hyaluronic acid, chondroitin sulfate, and heparin are presently used to treat a number of medical conditions. Pentosan polysulfate can be obtained by opening capsules of Elmiron® and removing the contents. Each capsule contains 100 mg of pentosan polysulfate, and various excipients. The contents of four such capsules can be used to prepare a medicament containing 400 mg of pentosan polysulfate.

By “physiologically acceptable acid, base, ester, and salt forms of such compounds” is meant a GAG in its acid, base, salt, or ester form, provided that, in the case of a salt form, the counter ion is physiologically acceptable to a human, and, in the case of esters, the organic group R in the ester (˜COOR) is a physiologically acceptable organic group. Esterification of one or more acid groups in a GAG molecule is accomplished using known organic chemistry techniques, e.g., reaction with an alcohol (ROH). An acid, base, and/or other catalyst can be employed to facilitate the reaction, so long as the disaccharide linkages are maintained.

A description of GAGs and related methods and compositions suitable for use in some embodiments of the present disclosure can be found in U.S. patent application Ser. No. 13/802,445, filed on Mar. 13, 2013, the entire content of which is incorporated herein by reference.

By “liposomal carrier” is meant a collection or plurality of liposomes. The liposomes can be made of any physiologically suitable phospholipid, glycolipid, derived lipid, and the like. Nonlimiting examples of suitable phospholipids include phosphatidylcholine, phosphatidyl-serine, phosphatidic acid, phosphatidylglycerin, phosphatidylethanolamine, phosphatidyl-inositol, sphingomyelin, dicetyl phosphate, lysophosphatidyl choline, and mixtures of such lipids, such as soybean phospholipids and egg yolk phospholipids, e.g., lecithin. Suitable glycolipids include cerebroside, sulphur-containing lipids, ganglioside, and the like. Suitable derived lipids include choleic acid, deoxycholic acid, and the like.

The liposomal carrier can be formed using any known method for forming liposomes, which can be loaded with a SVF, one or more glycosaminoglycans, and/or additional agents using any known method for forming and loading liposomes. Water, 70% to 100% alcohol, and similar solvents can be used to dilute the liposome preparation. Known methods for forming liposomes containing various agents are described, for example, in U.S. Pat. No. 4,235,871 to Papahadjopoulos, et al., and Oral Microbiology and Immunology, 1994, 2: 146-153, 30 the disclosures of which are incorporated herein by reference.

In one embodiment, the liposomes have a mean diameter of less than 200 nm, preferably less than 80 nm, more preferably less than 50 nm, as determined by, e.g., negative staining electron microscopy. Preparation of a substantially homogeneous population can be accomplished using conventional techniques, such as extrusion through a straight path or tortuous path-type filter Other methods of treating liposomes to form a homogenous size distribution include ultrasonic exposure (sonication), the French press technique, hydrodynamic shearing, homogenization using, for example, a colloid mill or Gaulin homogenizer, and microfluidization techniques. Microfluidization is a presently preferred method.

In one embodiment, a medicament comprising a SVF in a homogeneous liposomal carrier is prepared by intermittent homogenizing at 16,000 rpm with a handheld immersion blender for approximately 2 minutes.

The liposomal carrier according to an embodiment may be homogenized with a suitable amount of the SVF. Any suitable amount of the liposomal carrier and the SVF may be used. For example, in one embodiment, an amount of the liposomal carrier ranges from about 100 mg to about 300 mg. In another embodiment, for example, the amount of the liposomal carrier ranges from about 100 mg to about 200 mg. In some embodiments, the SVF comprises a number of cells ranging from about 1 million to about 200 million. In some embodiments, for example, the SVF comprises a number of cells ranging from about 5 million to about 50 million. In some embodiments, the SVF comprises about 5 million cells or more. In some embodiments, the SVF comprises about 5 million cells.

In some embodiments the medicament comprising the SVF in the homogeneous liposomal carrier is suitable for a daily dose.

In one embodiment, a medicament comprising a SVF and one or more additional agents, such as a glycosaminoglycan and/or a growth factor (e.g. a human growth factor), in a homogeneous liposomal carrier is prepared by intermittent homogenizing at 16,000 rpm with a handheld immersion blender for approximately 2 minutes.

The liposomal carrier according to an embodiment may be homogenized with a suitable amount of the SVF and a suitable amount of the one or more additional agents. In some embodiments the one or more additional agents include a GAG. In some embodiments, the one or more additional agents include a growth factor (e.g. a human growth factor). Any suitable amount of the liposomal carrier, the SVF, the GAG and/or the growth factor may be used. For example, in one embodiment, an amount of the liposomal carrier ranges from about 100 mg to about 300 mg. In another embodiment, for example, the amount of the liposomal carrier ranges from about 100 mg to about 200 mg. In other embodiments, the amount of the liposomal carrier is about 150 mg, for example, in embodiments where the liposomal carrier is used with pentosan polysulfate (PP) as the GAG. In some embodiments, the SVF comprises a number of cells ranging from about 1 million to about 200 million. In some embodiments, for example, the SVF comprises a number of cells ranging from about 5 million to about 50 million. In some embodiments, the SVF comprises about 5 million cells or more. In some embodiments, the SVF comprises about 5 million cells. In some embodiments, an amount the growth factor ranges from about 0.05 mg to about 0.5 mg. In some embodiments, for example, the amount of the growth factor ranges from about 0.1 mg to about 0.3 mg. In some embodiments, an amount of the GAG ranges from about 300 mg to about 600 mg. In some embodiments, for example, the amount of the GAG ranges from about 300 mg to about 500 mg. In other embodiments, the amount of the GAG is about 400 mg, for example, in embodiments where the GAG is pentosan polysulfate (PP). In some embodiments, a weight ratio of the liposomal carrier to the GAG is in a range of from about 1:6 to about 1:1. For example, in some embodiments the weight ratio of the liposomal carrier to the GAG is in a range of from about 1:5 to about 1:1. In another embodiment, the weight ratio of the liposomal carrier to the GAG is in a range of from about 1:5 to about 1:2. In another embodiment, the weight ratio of the liposomal carrier to the GAG is in a range of from about 1:3 to about 2:5. In another embodiment, the weight ratio of the liposomal carrier to the GAG is about 1.5:4, for example, in embodiments where the GAG is pentosan polysulfate (PP).

In some embodiments the medicament comprising the SVF and the one or more additional agents in the homogeneous liposomal carrier is suitable for a daily dose.

Microfluidization is described, for example, in U.S. Pat. No. 4,533,254 to Cook, et al., which is incorporated herein by reference. In one embodiment of a microfluidization procedure, the liposomal emulsion is forced at high pressure through a small diameter opening and splattered onto a wall and then collected. In sonication techniques, the raw materials for the liposomes, e.g., phospholipids, are combined with a SVF and additional agents, placed in a sonicator, and sonicated for a time, at a temperature and at a speed sufficient to obtain liposomes of the desired size.

The liposomes can be stored at reduced temperature, e.g., 40° F., until ready for use. In one embodiment, the liposomes, prior to administration, are treated to protect them against pH changes and micellization. In one embodiment, the liposomes are lyophilized. In another embodiment, the phospholipid (or any other constituent of the lipid wall) is treated with an additive, such as a crosslinking agent, prior to formation of the liposome.

Lyophilization may be accomplished by any method known in the art. Such procedures are disclosed, for example, in U.S. Pat. No. 4,880,836 to Janoff, et al., the disclosure of which is incorporated herein by reference. Lyophilization procedures can include the addition of a drying protectant to the liposome suspension to stabilize the liposome suspension so that the size and content of the liposomes are maintained during the drying procedure and through rehydration. Examples of drying agents include saccharide sugars, such as dextrose, sucrose, maltose, mannose, galactose, raffinose, trehalose lactose, and triose sugars, which can be added in amounts of about 5% to about 20%, more particularly, about 10%, by weight of the aqueous phase of the liposomal suspension. Manitol can be used in conjunction with any of the saccharides. Lyophilized liposomes can be reconstituted prior to use by adding water, saline, or other physiologically acceptable solvents.

A preservative such as BHT, EDTA, urea, albumin, dextran, or polyvinyl alcohol can be added to individual packaged doses for office use. Packaging should emphasize sterility but may also be designed to allow easy homogenization through the opening of the package, which can fit tightly around the tip of a hand blender (having, e.g., a 2 inch diameter steel head) to allow homogenization inside the package prior to dispensing. In one embodiment, a convenient port allows aspiration with a piston “catheter tip” syringe for instillation. This can be part of a kit. In one embodiment, a kit contains liposomes, separately packaged SVF and optionally a glucosaminoglycan and/or other agent(s), e.g., additional growth factors, DMSO or other small molecules, and a syringe/catheter tip. The SVF and any optional agent(s) can be added to the liposomes and the resulting combination homogenized immediately prior to use, then loaded into the syringe for administration to a patient.

In an alternate embodiment of the invention, a medicament comprising at least one growth factor in a liposomal carrier is provided, without a stromal vascular fraction. The liposomal carrier can be formed using any known method for forming liposomes, which can be loaded with at least one growth factor, or at least one growth factor and one or more additional agents, using any known method for forming and loading liposomes. The liposomes, liposomal carriers, and related methods are the same as those already described above with reference to the medicament comprising the SVF, the one or more glycosaminoglycans, and/or the additional agents. One or more growth factors is obtained (either from a commercial source or directly from a patient, and mixed with a liposomal carrier as described herein. The growth factor may be derived from a patient's stromal vascular fraction by removing stem cells and other components. An exemplary growth factor is HB-EGF. Other nonlimiting examples of growth factors are described above; a partial list is found in Table 1. Optionally, one or more additional agents are also included in the medicament. Nonlimiting examples include glycosaminoglycans (GAGs); small molecules like DMSO; and/or or other compounds used in the treatment of IC and/or having a physiological effect on stem cells. Nonlimiting examples of small molecules for the medicament are listed in Table 2.

In one embodiment, a medicament comprising at least one growth factor (e.g. a human growth factor) in a homogeneous liposomal carrier is prepared by intermittent homogenizing at 16,000 rpm with a handheld immersion blender for approximately 2 minutes.

The liposomal carrier according to an embodiment may be homogenized with a suitable amount of the at least one growth factor. Any suitable amount of the liposomal carrier and the at least one growth factor may be used. For example, in one embodiment, an amount of the liposomal carrier ranges from about 100 mg to about 300 mg. In some embodiments, for example, the amount of the liposomal carrier ranges from about 100 mg to about 200 mg. In some embodiments, an amount of the at least one growth factor ranges from about 0.05 mg to about 0.5 mg. In some embodiments, for example, the amount of the at least one growth factor ranges from about 0.1 mg to about 0.3 mg.

In some embodiments the medicament comprising the at least one growth factor in the liposomal carrier is suitable for a daily dose.

In one embodiment, a medicament comprising at least one growth factor (e.g. a human growth factor) and one or more additional agents, such as a glycosaminoglycan, in a homogeneous liposomal carrier is prepared by intermittent homogenizing at 16,000 rpm with a handheld immersion blender for approximately 2 minutes.

The liposomal carrier according to an embodiment may be homogenized with a suitable amount of the at least one growth factor and a suitable amount of the one or more additional agents. In some embodiments the one or more additional agents is a GAG. Any suitable amount of the liposomal carrier, the at least one growth factor, and the GAG may be used. For example, in one embodiment, an amount of the liposomal carrier ranges from about 100 mg to about 300 mg. In some embodiments, for example, the amount of the liposomal carrier ranges from about 100 mg to about 200 mg. In other embodiments, the amount of the liposomal carrier is about 150 mg, for example, in embodiments where the liposomal carrier is used with pentosan polysulfate (PP) as the GAG. In some embodiments, an amount of at least one growth factor ranges from about 0.05 mg to about 0.5 mg. In some embodiments, for example, the amount of the growth factor ranges from about 0.1 mg to about 0.3 mg. In some embodiments, an amount of the GAG ranges from about 300 mg to about 600 mg. In some embodiments, for example, the amount of the GAG ranges from about 300 mg to about 500 mg. In other embodiments, the amount of the GAG is about 400 mg, for example, in embodiments where the GAG is pentosan polysulfate (PP). In some embodiments, a weight ratio of the liposomal carrier to the GAG is in a range of from about 1:6 to about 1:1. For example, in some embodiments the weight ratio of the liposomal carrier to the GAG is in a range of from about 1:5 to about 1:1. In another embodiment, the weight ratio of the liposomal carrier to the GAG is in a range of from about 1:5 to about 1:2. In another embodiment, the weight ratio of the liposomal carrier to the GAG is in a range of from about 1:3 to about 2:5. In another embodiment, the weight ratio of the liposomal carrier to the GAG is about 1.5:4, for example, in embodiments where the GAG is pentosan polysulfate (PP).

In some embodiments the medicament comprising the at least one growth factor (e.g. a human growth factor) and the one or more additional agents in the homogeneous liposomal carrier is suitable for a daily dose.

In a second aspect of the invention a method of treating cystitis is provided and comprises intravesically administering a therapeutically effective amount of a medicament as described above, e.g., (1) an SVF provided in a liposomal carrier, (2) an SVF and one or more additional agents (as described above) in a liposomal carrier, (3) one or more growth factors provided in a liposomal carrier, or (4) one or more growth factors and one or more additional agents as described above, provided in a liposomal carrier. As used herein, the term “intravesically” and similar terms refer to administration of a medicament into the bladder, e.g. by a catheter. In a preferred embodiment, the SVF is derived from the patient's own adipose cells, so that the patient receives (e.g.) stem cells and/or growth factors only from his or her own body. Upon instillation, patients are instructed to retain the compound at least 30 minutes if possible. During this retention period, patients are instructed to turn 90 degrees every 5 minutes with exam table reverse trendelenberg (tilted head down approximately 30 degrees) and with reverse trendelenberg (tilted head up approximately 30 degrees).

In one embodiment of the invention, an additional dose of adult adipose-derived stem cells is administered to the patient intravenously, concurrently with the intravesical administration of the medicament (1, 2, 3, or 4). The intravenous dose is administered with or without liposomes and optionally contains one or more additional growth factors, GAGs, or physiologically acceptable small molecules to enhance the SVF. As with the intravesical instillation, autologous cells and growth factors are preferred so that the patient receives cells and/or growth factors from only his or her own body. Also, adult adipose-derived stem cells are preferred since embryonic stem cells have the potential to form rare tumors and their use has ethical considerations.

In still another embodiment of the invention, an additional dose of one or more growth factors is administered to the patient intravenously, concurrently with the intravesical administration of the medicament. The intravenous dose is administered with or without liposomes and optionally contains one or more additional agents, such as one or more GAGs or physiologically acceptable small molecules to enhance the growth factor. As with the intravesical instillation, autologous growth factors are preferred so that the patient receives growth factors from only his or her own body.

The following Examples are presented for illustrative purposes only and do not limit the scope of the invention.

### EXAMPLE 1

### Generic Protocol for Obtaining an SVF from a Patient

A. The patient will receive local anesthesia consisting of lidocaine 0.5% with epinephrine 1:400,000 with bicarbonate (HCO₃) 8.4% titrated to pH of 7.4 (generally 5 cc of HCO₃ in total volume of 60 cc).

B. The patient undergoes sterile prep.

C. The patient undergoes liposuction procedure utilizing the Lipokit machine (or another “syringe-type” liposuction procedure), fat processing unit (syringe) and 2.5-3 mm cannula.

D. The wound is protected with Bacitracin ointment, an adhesive bandage, and compressive bandage.

E. The SVF (ADSCs) are prepared in a closed system according to the following protocol:
1. If patient has adequate fat stores, then harvest 50 cc of fat and centrifuge at 2800 RPM×3 minutes. Loosen the plastic screw—this will allow free fatty acids to pass through filter within piston.
2. Pour out free triglycerides off the top and then tighten screw
3. Transfer bottom 1-2 cc of infranatant to Maxstem syringe use adaptor #TP-111 (there may be as many as 10% mesenchymal stromal cells in this patch and millions of WBCs and growth factors).
4. Eliminate the infranatant solution into waste basin
5. Transfer fat directly to Maxstem syringes (25 cc of fat) use adaptor #TP-111.
6. If inadequate fat stores, (e.g., small or thin patients) then harvest about 20-25 cc and do not centrifuge (add collagenase directly)—transfer to Maxstem syringe
7. Prepare 25 cc of collagenase solution to add to the 25 cc of fat.
Prepare collagenase solution:
   a. Add 20 cc of normal saline to 35 mg Liberase (175 Wunsch units total) (Roche Laboratories) (this is medical grade) and then transfer 1.5 cc to 12 or 13 3 cc syringes. Each syringe has about 12.5 Wunsch units. Leave a little air, cap, and place in freezer if not being used.
   b. Alternative—4 cc to 5 mg solution (25 Wunsch units) this is medical grade and GMP solution. Harvest 2 cc (12/5 Wunsch units each) into two 3 cc syringes.
   c. Draw up 23 cc of normal saline into a 30 cc syringe.
   d. Add 1.5 ml of Liberase solution to 23 cc solution in 30 cc syringe.
8. Add 25 cc collagenase (Liberase) solution to the fat in the Maxstem syringe using adaptor #TP-113.
9. Shake by hand several times, then place in sterile stainless steel incubator bin and into incubator for 30 minutes at 38° C.
10. Place in centrifuge with screw still tight and spin at 200 g for 4 minutes.
11. Remove and keep upright
12. Remove plastic screw from piston
13. Insert tube #TP-109 by screwing it into the piston (snug but don't over tighten). Attach the original, now empty, liposuction syringe to the tube or can use a 60 cc syringe and adaptor #FG-103.
14. Push down on the piston and the supernatant fat and fluid will collect in the upper syringe. Can stop when piston hits bottom, but recommend leaving about 5 cc solution and then stopping.
15. Unscrew waste removal syringe from the tube #TP-109 and connect wash syringe with FG-103 adaptor with 50 cc of D5LR to tube and pull up while gently pushing plunger. The D5LR will then top off the syringe. This is the wash solution used to wash out residual collagenase. Remove the tube and replace the screw. Tighten so it's snug (but don't over tighten as it will be hard to remove).
16. Wash is performed at same centrifugation of 200 g for 4 minutes
17. Wash solution is refilled from the sterile tubing connected to the D5LR.
18. Repeat steps 15-17 two more times so that after the original supernatant was removed, there are a total of three washes.
19. After the final wash, remove the syringe, remove the screw from the piston and insert the tube #TP-109 with a disposal (waste) syringe with adaptor FG-103.
20. Force the piston down removing all but the last 5 or 6 cc's of the infranatant solution.
21. Remove the tube (TP-109) and replace the screw securing it tight.
22. Prepare a 10 cc syringe with 1 cc of wash (D5LR) solution
23. Remove the bottom cap and secure the 10 cc luerlok syringe with 1 cc solution to adaptor FG-103 connected to TP-111 (no filter).
24. Once secure, loosen the piston screw inside the large Maxstem syringe and then draw off the bottom 1 cc of solution into the 10 cc luerlok syringe.
25. Tighten screw in piston again.
26. Secure adaptors TP-111 with 100 micron mesh between it and adaptor FG-103 to 10 cc luerlok and to the Maxstem syringe.
27. Loosen piston screw again.
28. Draw off remaining filtered solution through the filtered adaptors.
29. Add the infranatant (original unfiltered thick infranatant fluid) to the filtered 10 cc syringe. This is done through adaptors FG-103 and TP-113—a 100 micron filter is secured between these adaptors first. Keep this (unfiltered syringe below the filtered syringe and push upward while pulling back on the filtered syringe plunger. This leaves the debris dependent and adds extra cells (previously discarded) back into the SVF syringe.
30. Save a drop for cell counting later.
31. Cells can be given in a number of ways or dilutions (no further dilution or add saline to use in other conditions). It's generally believed that 5 million cells is an adequate dose for most degenerative conditions.
32. One syringe should generally yield 10-50 million cells. This can then be used in a single dose for intravesical administration or divided into and intravenous and an intravesical dose.
33. If used via an intravenous line—place cells into 100 cc of normal saline. Drip through filtered IV tube line over 30 minutes.

### EXAMPLE 2

### Preparation of a Medicament Containing a SVF in a Liposomal Carrier

A. 2,250 ml. of water (double distilled) is charged to a beaker (keep cool in ice bath, etc) and a nitrogen sparge is set for at least 30 minutes.
B. Add 225 grams of maltose (Sigma M5885) to the water and mix until dissolved. Keep the nitrogen sparge going. Maintain pH of 4.81 by adding acetic acid as needed.
C. In another beaker, 10.59 grams of egg phosphatidylcholine (EPC) (Sigma or equivalent substitute) is combined with 8.38 ml. of ethanol (anhydrous, Sigma E3884) and mixed until dissolved. To this add 67.5 mg. of BHT and mix until dissolved. To this mixture add 3.5 ml. of SVF, and mix until dissolved. Use the remaining 4.19 ml. of ethanol to rinse any remaining contents of the weighing container into the mixture.
D. Draw the ethanol solution into a 10 ml. syringe and add to the maltose solution over 11 minutes with continued nitrogen sparge. Keep pH<7.0 (goes into Microfluidizer at pH 4.81). Measure. Hand blade mixture. Keep everything cool at about 1.5 degrees C.
E. Add to Microfluidizer. Four passes through a M-110Y high-pressure pneumatic microfluidizer (Microfluidics, Newton, Mass.), with the pressure set at 16,000 psi. Keep the Microfluidizer homogenization chamber cooled (ice bath or other coolant) as the passage through the small aperture causes a microsecond of heating to occur.

| Ingredient | Quantity | |
|---|---|---|
| EPC: | 10.59 | g |
| Maltose: | 225 | g |
| Ethanol: | 12.57 | ml |
| BHT: | 67.5 | mg |
| SVF | 3.5 | ml |
| (USP) Water: | 2,250 | ml |

A multiplier of 10.158 is used for the scale-up.

The above protocol can be used to encapsulate up to about 2 grams (2,160 mg) of SVF and one or more additional components.

At 16,000 psi in the microfluidizer, localized heating can occur. The melting point of maltose is 102-103° C., so it is important to cool the mixing chamber using an ice bath or other cooling means.

### EXAMPLE 3

### Instillation of SVF-Containing-Liposomes in an Interstitial Cystitis Patient

100 cc of liposomes containing a SVF are placed in a 500 cc metal mixing bowel on a sterile field, and 100 cc of sterile water are slowly added as the mixture is homogenized over 1-2 minutes at 16000 rpm. The solution is then slowly instilled through a silicone catheter into a patient's bladder using a 70 cc piston syringe with a “catheter tip.”

IC patients treated with an instillation containing SVF in liposomes were able to retain the compound in their bladders for up to 45 minutes. Preliminary studies on two patients treated with a single outpatient instillation of SVF-in-liposomes showed no negative side effects. No infection, pain, bleeding, or system side effects were noted. Both patients reported improvement in their symptoms as described in their Pelvic Urgency/Frequency scores and the O'Leary-Sant quality of life scores. O'Leary-Sant scores and Pelvic Urgency Frequency scores are subjective outcome tools which can be used to evaluate efficacy of a treatment for IC.

The O'Leary-Sant scores, as used in this example, can be used for evaluating whether a patient has IC or for evaluating patients with IC before, during, and/or after a treatment for IC. The O'Leary-Sant test scores include a symptom index and a problem index. The symptom index measures urgency and pain in patients being evaluated for IC. The problem index measures a degree to which patients experience each symptom. IC is typically diagnosed when a score of greater than 6 is provided in each symptom index.

The Pelvic Urgency Frequency scores, as used in this example, can also be used for evaluating whether a patient has IC or for evaluating patients with IC before, during, and/or after a treatment for IC. Additionally, Pelvic Urgency Frequency scores can be used for evaluating pelvic pain, and particularly, chronic pelvic pain in a patient. Pelvic Urgency Frequency scores focus on urgency and frequency issues in IC, and pain and symptoms associated with sexual intercourse. Here, a score of greater than 5 indicates approximately a 55% chance of IC, while a score of greater than 10 indicates approximately a 74% chance of IC.

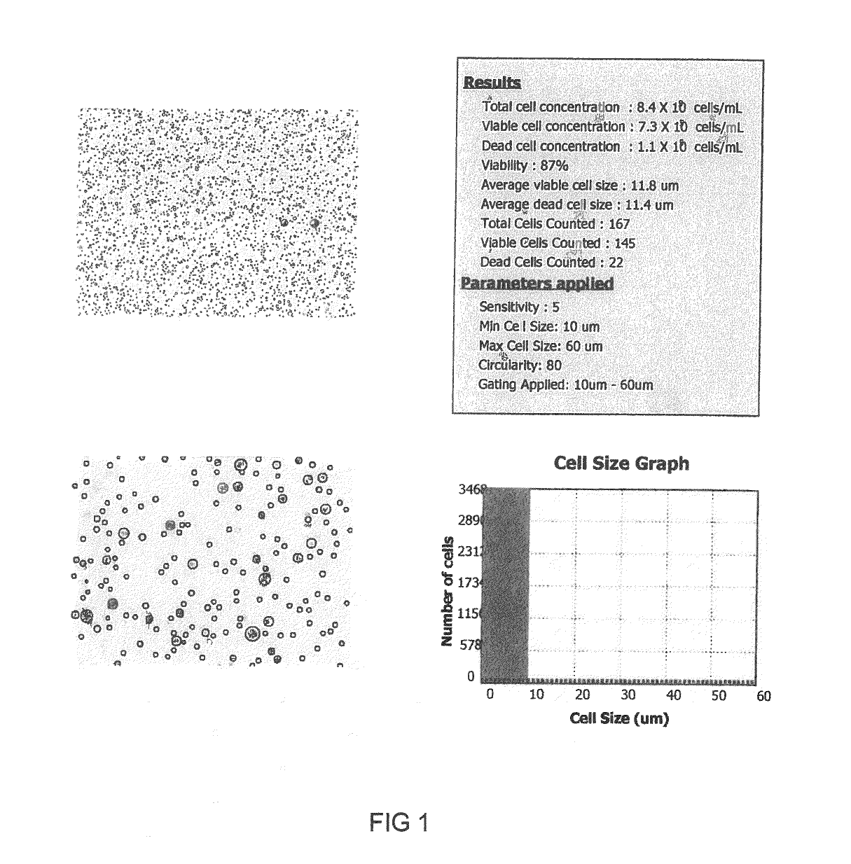

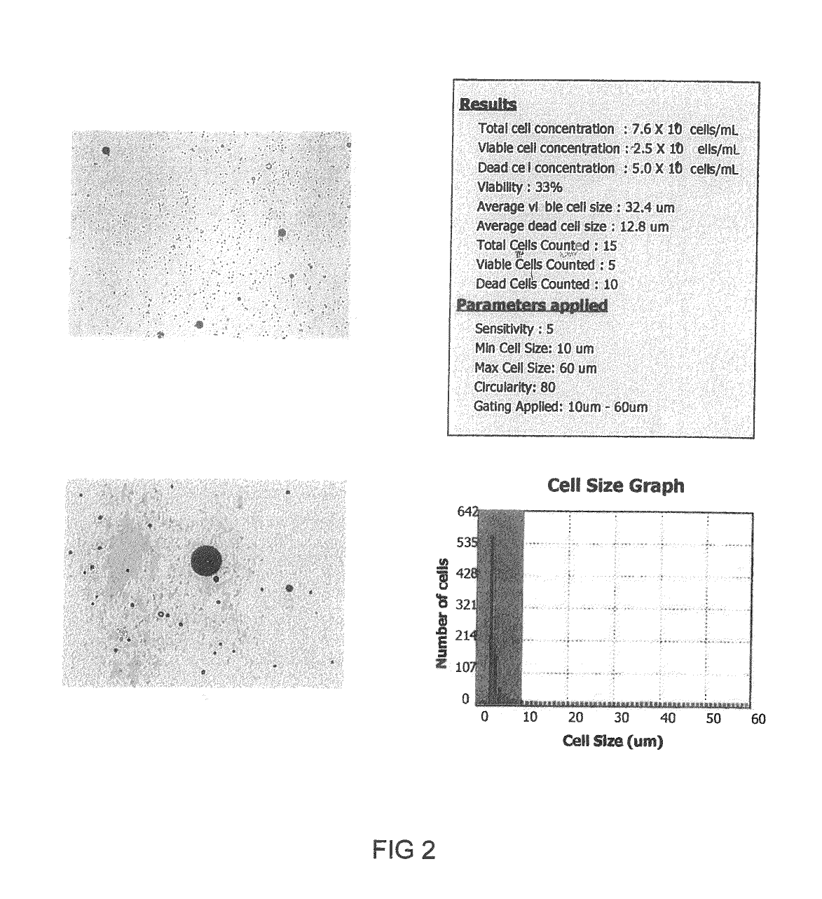

FIGS. 1 and 2 are print-outs from an Invitrogen™ cell counter, showing digital images and related data from a patient's stromal vascular fraction (SVF) containing mesenchymal stem cells. FIG. 1 corresponds to the SVF (diluted 5:1 with normal saline) prior to contact with liposomes. The mesenchymal stem cells are stained with trypan blue 0.4% to check viability. the cell counter has been set to measure cells between 10 and 60 microns in size.

FIG. 2 shows the same cells after being mixed under sterile conditions with liposomes, according to the invention. Cell appearance is obscured by liposomes, which either adhere to the cells or encapsulate them.

From the preceding disclosure, various modifications and alternate embodiments of the invention will be apparent to persons skilled in the art to which the invention pertains. All such modifications and embodiments are within the scope of the invention, which is limited only by the appended claims and equivalents thereof.

What is claimed is:

1. A medicament for treating cystitis, comprising a stromal vascular fraction (SVF) in a liposomal carrier.

2. A medicament as recited in claim 1, wherein the SVF is obtained from a patient by liposuction.

3. A medicament as recited in claim 1, wherein the liposomal carrier comprises liposomes having a mean diameter of 200 nm or less or having a mean diameter of 50 nm or less.

4. A medicament as recited in claim 1, further comprising one or more additional agents selected from the group consisting of growth factors, glycosaminoglycans, and physiologically acceptable small molecules.

5. A medicament as recited in claim 4, wherein said one or more additional agents comprises at least one selected from the group consisting of a glycosaminoglycan (“GAG”) and DMSO.

6. A medicament as recited in claim 5, wherein the GAG is selected from the group consisting of chondroitins, dermatans, heparans, heparins, hyaluronans, keratans, pentosans, physiologically acceptable acid, base, ester, and salt forms of such compounds, and mixtures thereof.

7. A medicament as recited in claim 5, wherein the GAG comprises at least one selected from the group consisting of sodium pentosan polysulfate, chondroitin sulfate, hyaluronic acid, a physiologically acceptable ester of hyaluronic acid, and a physiologically acceptable salt hyaluronic acid.

8. A medicament for treating cystitis, comprising at least one human growth factor in a liposomal carrier.

9. A medicament as recited in claim 8, wherein the at least one human growth factor comprises a heparin-binding epidermal growth factor.

10. A medicament as recited in claim 8, further comprising one or more additional agents selected from the group consisting of glycosaminoglycans and physiologically acceptable small molecules.

11. A method of treating cystitis, comprising:
intravesically administering to a patient a therapeutically effective dose of a medicament comprising a stromal vascular fraction (“SVF”) in a liposomal carrier.

12. A method as recited in claim 11, wherein the SVF has been obtained from the patient's own adipose cells.

13. A method as recited in claim 11, wherein the liposomal carrier comprises liposomes having a mean diameter of 50 nm or less.

14. A method as recited in claim 11, wherein the cystitis is selected from the group consisting of interstitial cystitis, infectious cystitis, radiation cystitis, and chemical cystitis.

15. A method as recited in claim 11, wherein the medicament further comprises one or more additional agents selected from the group consisting of growth factors, glycosaminoglycans, and DMSO or other physiologically acceptable small molecules.

16. A method as recited in claim 15, wherein the GAG is selected from the group consisting of chondroitins, dermatans, heparans, heparins, hyaluronans, keratans, pentosans, physiologically acceptable acid, base, ester, and salt forms of such compounds, and mixtures thereof.

17. A method as recited in claim 15, wherein the GAG comprises at least one selected from the group consisting of sodium pentosan polysulfate, chondroitin sulfate, hyaluronic acid, a physiologically acceptable ester of hyaluronic acid, and a physiologically acceptable salt hyaluronic acid.

18. A method as recited in claim 15, wherein said one or more additional agents comprises heparin-binding epidermal growth factor.

19. A method as recited in claim 11, further comprising intravenously administering an additional therapeutically effective dose of a stromal vascular fraction, concurrently with the intravesical administration.

20. A method as recited in claim 19, wherein the additional dose of a stromal vascular fraction is in a liposomal carrier.

21. A method as recited in claim 19, wherein the additional dose of a stromal vascular fraction also comprises one or more agents selected from the group consisting of GAGs, growth factors, and physiologically acceptable small molecules.

22. A method as recited in claim 11, further comprising one selected from the group consisting of intravenously administering a therapeutically effective dose of at least one human growth factor, and intravenously administering a therapeutically effective dose of at least one human growth factor in a liposomal carrier.

23. A method of treating cystitis, comprising:
intravesically administering to a patient a therapeutically effective dose of a medicament comprising at least one human growth factor in a liposomal carrier.

24. A method as recited in claim 23, wherein the medicament further comprises one or more agents selected from the group consisting of GAGs, growth factors, and physiologically acceptable small molecules.

25. A method as recited in claim 23, further comprising one selected from the group consisting of:
intravenously administering an additional therapeutically effective dose of at least one human growth factor,
intravenously administering an additional therapeutically effective dose of at least one human growth factor in a liposomal carrier;
intravenously administering an additional therapeutically effective dose of at least one human growth factor and one or more agents selected from the group consisting of GAGs and physiologically acceptable small molecules,
intravenously administering an additional therapeutically effective dose of at least one human growth factor and one or more agents selected from the group consisting of GAGs and physiologically acceptable small molecules, in a liposomal carrier.
